(19) United States
(12) Patent Application Publication
(10) Pub. No.: US 2021/0207171 A1
(43) Pub. Date: Jul. 8, 2021

(54) FORMULATIONS FOR SIMIAN ADENOVIRAL VECTORS HAVING ENHANCED STABILITY

(71) Applicant: GLAXOSMITHKLINE BIOLOGICALS SA, Rixensart (BE)

(72) Inventors: Frédéric Stéphane MATHOT, Rixensart (BE); Mathieu VASSELLE, Rixensart (BE)

(73) Assignee: GLAXOSMITHKLINE BIOLOGICALS SA, Rixensart (BE)

(21) Appl. No.: 17/251,518

(22) PCT Filed: Jun. 25, 2019
(86) PCT No.: PCT/IB2019/055347

§ 371 Date: Dec. 11, 2020

(30) Foreign Application Priority Data
Jun. 26, 2018 (EP) 18179977.6

(51) Int. Cl. C12N 15/86; C07K 14/765; A61K 47/26

(52) U.S. Cl. CPC C12N 15/86; C12N 2710/10043; A61K 47/26; C07K 14/765

(57) ABSTRACT

The invention relates to liquid formulations of simian adenoviruses and methods for obtaining the formulations. The formulations comprise an amorphous sugar, Vitamin E succinate and recombinant human serum albumin in a buffered solution.

### FIELD OF THE INVENTION

The invention relates to the formulation of simian adenoviral vectors in liquid compositions, their formulations and methods of using the compositions.

### BACKGROUND

Adenoviral vectors represent a prophylactic or therapeutic protein delivery platform whereby a nucleic acid sequence encoding a prophylactic or therapeutic protein is incorporated into the adenoviral genome, which is brought to expression when the adenoviral particle is administered to the treated subject. It has been a challenge in the art to develop stabilizing formulations for the adenoviral vectors which allow storage at acceptable storage temperatures with a considerable shelf life.

Stabilizing simian adenovirus by lyophilization has been reported (WO 2017/013169; BioProcess Int. (2018) 16:26). Stabilizing liquid formulations have been reported for human adenoviral vectors (J. Pharm. Sci. (2004) 93:2458-2475). A stable liquid human adenovirus formulation was reported with a loss of approximately 0.5 log infectivity at 4° C. over 24 months, with a shelf life specification, based on the minimum required dose for biological efficiency according to ICH guidelines, of a 0.9 log loss (Eur. J. Biopharm. (2018) 129:215). However, there remains a need in the art for liquid formulations that preserve the stability of simian adenoviral vectors. Such formulations would decrease vaccine waste due to disruption of the cold chain and facilitate vaccine production, shipment, storage and patient compliance.

### SUMMARY OF THE INVENTION

The inventors surprisingly found that tocopherol and recombinant human serum albumin greatly increased the stability of simian adenovirus when incorporated into a composition comprising an amorphous sugar. The invention therefore provides a stable aqueous liquid formulation for simian adenoviruses comprising tocopherol, recombinant human serum albumin and at least one amorphous sugar. Adenoviruses formulated as described herein are thermostable. The invention further provides methods of using the stabilized recombinant simian adenoviruses to confer prophylactic immunity and to act as therapeutic vectors by delivering a transgene to a human subject.

### BRIEF DESCRIPTION OF THE DRAWINGS

FIG. 1: Effect of osmolality and sugar composition on stability. Stability was measured by PICOGREEN following exposure of the virus to a temperature of 4° C. for two and a half months or 30° C. for one month.

FIG. 2: Effect of trehalose, sucrose, VES and rHSA on stability. “BDS-ADVAR” refers to 10 mM Tris pH 8.5, 5 mM NaCl, 10 mM histidine, 0.025% polysorbate 80 (w/v) and 1 mM MgCl₂. Stability was measured by analytical HPLC after exposure of the simian adenovirus to 4° C. or 25° C. for three weeks.

FIG. 3A: Real time stability at 4° C. Simian adenovirus in 10 mM Tris pH 8.5, 5 mM NaCl, 10 mM histidine, 0.024% polysorbate 80 (w/v), 1 mM MgCl₂, 15% trehalose (w/v), 2% sucrose (w/v), 0.05 mM VES and 0.1% rHSA (w/v) at a concentration (comprising a 10% overage) of 1.1×10¹¹ pu/ml (virus particle units per milliliter) over a six-month period measured by two independent analytical HPLC methods (solid and dotted lines).

FIG. 3B: Real time stability at 4° C. Simian adenovirus in 10 mM Tris pH 8.5, 5 mM NaCl, 10 mM histidine, 0.024% polysorbate 80 (w/v), 1 mM MgCl₂, 16% sucrose and 0.05 mM VES at a concentration (comprising a 10% overage) of 1.1×10¹¹ pu/ml (virus particle units per milliliter) over a six-month period measured by two independent analytical HPLC methods (solid and dotted lines).

FIG. 3C: Real time stability at 4° C. Simian adenovirus in 10 mM Tris pH 8.5, 5 mM NaCl, 10 mM histidine, 0.024% polysorbate 80 (w/v), 1 mM MgCl₂, 15% trehalose (w/v), 2% sucrose (w/v), 0.05 mM VES and 0.1% rHSA (w/v) at a concentration (comprising a 10% overage) of 1.1×10¹⁰ pu/ml (virus particle units per milliliter) over a six-month period measured by two independent analytical HPLC methods (solid and dotted lines).

FIG. 4: Immunogenicity in mice determined by IFN gamma ELIspot and compared to lyophilized simian adenovirus.

FIG. 5: Forced degradation of simian adenovirus via metal and light oxidation. Stability was measured by analytical high performance liquid chromatography (HPLC) after exposure of the virus to metal and light oxidation under Acceleration Oxidation Test (AOT) conditions.

### DETAILED DESCRIPTION

The inventors found that formulations developed for stabilizing human adenoviral vectors could not successfully be applied to all adenoviral vectors, e.g. simian adenoviral vectors, rendering them stable, thus useful for prophylaxis or therapy. For example, A195 buffer (10 mM Tris pH 7.4, 10 mM histidine, 75 mM NaCl, 5% sucrose, 0.02% polysorbate 80, 0.1 mM EDTA, 1 mM MgCl₂), which is used to stabilize human adenovirus, is not suitable for stabilizing simian adenoviruses (BioProcess Int (2018) 16:26). The present invention describes compositions of simian adenovirus wherein the structural integrity and functionality of the adenoviral particle is better protected or maintained.

### Simian Adenoviruses

Adenoviruses are nonenveloped viruses with an icosahedral capsid that contains a double stranded DNA genome. The capsid comprises three major proteins, hexon (II), penton base (III) and a knobbed fiber (IV), along with a number of other minor proteins, VI, VIII, IX, IIIa and IVa2 that mediate the early stages of adenoviral infection. The hexon accounts for the majority of the structural components of the capsid, which consists of 240 trimeric hexon capsomeres and 12 penton bases. The hexon has three conserved double barrels, while the top has three towers, each tower containing a loop from each subunit that forms most of the capsid. The base of the hexon is highly conserved between adenoviral serotypes, while the surface loops are variable. The penton is another adenoviral capsid protein that forms a pentameric base to which the fiber attaches. The trimeric fiber protein protrudes from the penton base at each of the 12 vertices of the capsid and is a knobbed rod-like structure. The primary role of the fiber protein is the tethering of the viral capsid to the cell surface via the interaction of the knob region with a cellular receptor, and variations in the flexible shaft as well as knob regions of fiber are characteristic of the different serotypes.

By “simian” is meant any member of the infraorder Simiiformes. It includes Platyrrhini (New World monkeys) and Catarrhini (Old World monkeys and apes). It includes bonobos, capuchins, chimpanzees, gibbons, gorillas, great apes, howler monkeys, marmosets, orangutans, owl monkeys, sakis, spider monkeys, squirrel monkeys, tamarinds, titis, uakaris and woolly monkeys. Numerous adenoviruses have been isolated from simians such as chimpanzees, bonobos, rhesus macaques and gorillas. Vectors derived from these adenoviruses have been shown to induce strong immune responses to encoded transgenes (Sci Transl Med (2012) 4:1; Virol (2004) 324: 361; J Gene Med (2010) 13:17). Also, simian adenoviruses demonstrate a relative lack of cross-neutralizing antibodies compared to human adenoviruses in the human population.

Adenoviruses can be used as vectors to deliver desired RNA or protein sequences, for example heterologous sequences, for in vivo expression. An adenoviral vector may include any genetic element including naked DNA, a phage, transposon, cosmid, episome, plasmid, or virus. Such vectors contain DNA of the simian adenovirus and an expression cassette. By “expression cassette” (or “minigene”) is meant the combination of a selected heterologous gene (“transgene” or “gene of interest”) and other regulatory elements necessary to drive translation, transcription and/or expression of the gene product in a host cell. In embodiments of the invention, the simian adenoviral vector may comprise one or more of a promoter, an enhancer, and a reporter gene.

Adenoviral vectors of the invention may contain simian adenoviral DNA. In one embodiment, the adenoviral vector of the invention is derived from a nonhuman simian adenovirus, also referred to as a “simian adenovirus.” Numerous adenoviruses have been isolated from nonhuman simians such as chimpanzees, bonobos, rhesus macaques, orangutans and gorillas. Vectors derived from these adenoviruses can induce strong immune responses to transgenes encoded by these vectors. Certain advantages of vectors based on nonhuman simian adenoviruses include a relative lack of cross-neutralizing antibodies to these adenoviruses in the human target population, thus their use overcomes the pre-existing immunity to human adenoviruses.

Adenoviral vectors of the invention may be derived from a non-human simian adenovirus, e.g., from chimpanzees (Pan troglodytes), bonobos (Pan paniscus), gorillas (Gorilla gorilla), orangutans (Pongo abelii and Pongo pygnaeus) and macaques (any of the species of the genus Macaca). They include adenoviruses from Group B, Group C, Group D, Group E and Group G. Vectors may include, in whole or in part, a nucleotide encoding the fiber, penton or hexon of a non-human adenovirus.

In addition to the transgene, the expression cassette also may include conventional control elements which are operably linked to the transgene in a manner that permits its transcription, translation and/or expression in a cell transfected with the adenoviral vector. As used herein, “operably linked” sequences include both expression control sequences that are contiguous with the gene of interest and expression control sequences that act in trans or at a distance to control the gene of interest.

Regulatory elements, i.e., expression control sequences, include appropriate transcription initiation, termination, promoter and enhancer sequences; efficient RNA processing signals such as splicing and polyadenylation (poly A) signals including rabbit beta-globin polyA; tetracycline regulatable systems, microRNAs, posttranscriptional regulatory elements e.g., WPRE, posttranscriptional regulatory element of woodchuck hepatitis virus); sequences that stabilize cytoplasmic mRNA; sequences that enhance translation efficiency (e.g., Kozak consensus sequence); sequences that enhance protein stability; and when desired, sequences that enhance secretion of an encoded product.

A “promoter” is a nucleotide sequence that permits the binding of RNA polymerase and directs the transcription of a gene. Typically, a promoter is located in a non-coding region of a gene, proximal to the transcriptional start site. Sequence elements within promoters that function in the initiation of transcription are often characterized by consensus nucleotide sequences. Examples of promoters include, but are not limited to, promoters from bacteria, yeast, plants, viruses, and mammals, including simians and humans. A great number of expression control sequences, including promoters which are internal, native, constitutive, inducible and/or tissue-specific, are known in the art and may be utilized.

A “posttranscriptional regulatory element,” as used herein, is a DNA sequence that, when transcribed, enhances the expression of the transgene(s) or fragments thereof that are delivered by viral vectors of the invention. Posttranscriptional regulatory elements include, but are not limited to, the Hepatitis B Virus Posttranscriptional Regulatory Element (HPRE) and the Woodchuck Hepatitis Posttranscriptional Regulatory Element (WPRE). The WPRE is a tripartite cis-acting element that has been demonstrated to enhance transgene expression driven by certain, but not all promoters.

The transgene encoded by the adenoviral vector will typically encode a product useful in biology or medicine, such as a therapeutic or immunogenic protein, an enzyme, or an RNA. Desirable RNA molecules include tRNA, dsRNA, ribosomal RNA, catalytic RNAs, RNA aptamers, and antisense RNAs. One example of a useful RNA sequence is a sequence which extinguishes expression of a targeted nucleic acid sequence in the treated subject. The transgene may encode a polypeptide or protein used for treatment, e.g., of genetic deficiencies, as a cancer therapeutic or vaccine, for induction of an immune response, and/or for prophylactic vaccine purposes. Particularly the polypeptide or protein is an antigen.

As used herein, induction of an immune response refers to the ability of a protein, also known as an “antigen” or “immunogen,” to induce a T cell and/or a humoral immune response to the protein. Unless otherwise indicated, “therapy” or “therapeutic” may relate to either or both preventive and curative therapy.

The transgene may be used for prophylaxis or treatment, e.g., as a vaccine for inducing an immune response, to correct genetic deficiencies by correcting or replacing a defective or missing gene, or as a cancer therapeutic. Particularly, the immune response is a protective immune response.

Compositions of the invention may be immunogenic compositions. Optionally, a mixture or composition of the invention may be formulated to contain other components, including, e.g., further immunogen(s), e.g. polypeptide antigen(s), and/or adjuvants. In an embodiment, the invention provides a vaccine comprising an adjuvant. Such an adjuvant can be administered with a priming DNA vaccine encoding an antigen to enhance the antigen-specific immune response compared with the immune response generated upon priming with a DNA vaccine encoding the antigen only. Alternatively, such an adjuvant can be administered with a polypeptide antigen which is administered in a regimen involving the adenoviral vectors of the invention.

The immune response elicited by the transgene may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing cytokines, e.g. interferon gamma (IFN gamma), tumor necrosis factor alpha (TNF alpha) and/or interleukin 2 (IL2). Alternatively, or additionally, the antigen specific T cell response comprises a CD8+ T cell response, such as a response involving CD8+ T cells expressing cytokines, e.g., IFN gamma, TNF alpha and/or IL2.

Thus, a composition of the invention is for use in prophylactic (i.e., immunogenic or preventive) or therapeutic treatment of a subject, as a result of the action of the transgene encoded by the adenoviral vector. Compositions of the invention are suitable for intramuscular injection.

The methods of the invention may induce a protective or therapeutic immune response to a disease. In an embodiment, the disease is an infectious or an oncogenic disease. In an embodiment the protective immune response is achieved by immunizing or vaccinating a subject against a pathogen. The invention may therefore be applied for the prophylaxis, treatment or amelioration of diseases due to infection by pathogens, e.g., viruses, bacteria, fungi, parasitic microorganisms or multicellular parasites which infect human and non-human vertebrates, or from a cancer cell or tumor cell. Immunogens may be selected from a variety of viruses, bacteria, fungi, parasitic microorganisms or multicellular parasites.

A “thermostable” adenovirus formulation is one in which the adenovirus can resist irreversible change in its physical or chemical structure without losing its characteristic properties at moderately high relative temperatures.

The term “replication-defective” or “replication-incompetent” adenovirus refers to an adenovirus that is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc., or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF4, E4 ORF3, E4 ORF2 and/or E4 ORF1). Suitably, E1 and optionally E3 and/or E4 are deleted. If deleted, the aforementioned deleted gene region will suitably not be considered in the alignment when determining percent identity with respect to another sequence.

For the purposes of comparing two closely-related polynucleotide or polypeptide sequences, the “% identity” between a first sequence and a second sequence may be calculated using an alignment program, such as BLAST@ (available at blast.ncbi.nlm.nih.gov, last accessed 9 Mar. 2015) using standard settings. The % identity is the number of identical residues divided by the number of residues in the reference sequence, multiplied by 100. The % identity figures referred to above and in the claims are percentages calculated by this methodology. An alternative definition of % identity is the number of identical residues divided by the number of aligned residues, multiplied by 100.

Alternative methods include using a gapped method in which gaps in the alignment, for example deletions in one sequence relative to the other sequence, are accounted for in a gap score or a gap cost in the scoring parameter. For more information, see the BLAST@ fact sheet available at ftp.ncbi.nlm.nih.gov/pub/factsheets/HowTo_BLASTGuide.pdf, last accessed on 9 Mar. 2015.

Sequences that preserve the functionality of the polynucleotide or a polypeptide encoded thereby are likely to be more closely identical. Polypeptide or polynucleotide sequences are said to be the same as or identical to other polypeptide or polynucleotide sequences, if they share 100% sequence identity over their entire length.

A “difference” between sequences refers to an insertion, deletion or substitution of a single amino acid residue in a position of the second sequence, compared to the first sequence. Two polypeptide sequences can contain one, two or more such amino acid differences. Insertions, deletions or substitutions in a second sequence which is otherwise identical (100% sequence identity) to a first sequence result in reduced percent sequence identity. For example, if the identical sequences are nine amino acid residues long, one substitution in the second sequence results in a sequence identity of 88.9%. If the identical sequences are 17 amino acid residues long, two substitutions in the second sequence results in a sequence identity of 88.2%. If the identical sequences are seven amino acid residues long, three substitutions in the second sequence results in a sequence identity of 57.1%. If first and second polypeptide sequences are nine amino acid residues long and share six identical residues, the first and second polypeptide sequences share greater than 66% identity (the first and second polypeptide sequences share 66.7% identity). If the first and second polypeptide sequences are 17 amino acid residues long and share 16 identical residues, the first and second polypeptide sequences share greater than 94% identity (the first and second polypeptide sequences share 94.1% identity). If the first and second polypeptide sequences are seven amino acid residues long and share three identical residues, the first and second polypeptide sequences share greater than 42% identity (the first and second polypeptide sequences share 42.9% identity).

Alternatively, for the purposes of comparing a first, reference polypeptide sequence to a second, comparison polypeptide sequence, the number of additions, substitutions and/or deletions made to the first sequence to produce the second sequence may be ascertained. An addition is the addition of one amino acid residue into the sequence of the first polypeptide (including addition at either terminus of the first polypeptide).

A substitution is the substitution of one amino acid residue in the sequence of the first polypeptide with one different amino acid residue. A deletion is the deletion of one amino acid residue from the sequence of the first polypeptide (including deletion at either terminus of the first polypeptide). For the purposes of comparing a first, reference polynucleotide sequence to a second, comparison polynucleotide sequence, the number of additions, substitutions and/or deletions made to the first sequence to produce the second sequence may be ascertained. An addition is the addition of one nucleotide residue into the sequence of the first polynucleotide (including addition at either terminus of the first polynucleotide). A substitution is the substitution of one nucleotide residue in the sequence of the first polynucleotide with one different nucleotide residue. A deletion is the deletion of one nucleotide residue from the sequence of the first polynucleotide (including deletion at either terminus of the first polynucleotide). Suitably substitutions in the sequences of the present invention may be conservative substitutions. A conservative substitution comprises the substitution of an amino acid with another amino acid having a chemical property similar to the amino acid that is substituted (see, for example, Stryer et al, Biochemistry, 5th ed. 2002, pages 44-49). Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is preferably selected from the group consisting of arginine, histidine, and lysine.

An acidic amino acid is preferably aspartate or glutamate. An aromatic amino acid is preferably selected from the group consisting of phenylalanine, tyrosine and tryptophan. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

Alternatively or additionally, the cross-protective breadth of a vaccine construct can be increased by comprising a medoid sequence of an antigen. By “medoid” is meant a sequence with a minimal dissimilarity to other sequences. Alternatively or additionally, a vector of the invention comprises a medoid sequence of a protein or immunogenic fragment thereof. Alternatively or additionally, the medoid sequence is derived from a natural viral strain with the highest average percent of amino acid identity among all related protein sequences annotated in the NCBI database.

As a result of the redundancy in the genetic code, a polypeptide can be encoded by a variety of different nucleic acid sequences. Coding is biased to use some synonymous codons, i.e., codons that encode the same amino acid, more than others. By “codon optimized” it is meant that modifications in the codon composition of a recombinant nucleic acid are made without altering the amino acid sequence. Codon optimization has been used to improve mRNA expression in different organisms by using organism-specific codon-usage frequencies.

In addition to, and independently from, codon bias, juxtaposition of codons in open reading frames is not random and some codon pairs are used more frequently than others. This codon pair bias means that some codon pairs are overrepresented and others are underrepresented. By “codon pair optimized,” it is meant that modifications in the codon pairing are made without altering the amino acid sequence of the individual codons. Constructs of the invention can comprise a codon optimized nucleic acid sequence and/or a codon pair optimized nucleic acid sequence

The term “replication-competent” adenovirus refers to an adenovirus which can replicate in a host cell in the absence of any recombinant helper proteins comprised in the cell. Suitably, a “replication-competent” adenovirus comprises intact structural genes and the following intact or functionally essential early genes: E1A, E1B, E2A, E2B and E4.

In an embodiment of the invention, the vector is a functional or an immunogenic derivative of an adenoviral vector. By “derivative of an adenoviral vector” is meant a modified version of the vector, e.g., one or more nucleotides of the vector are deleted, inserted, modified or substituted.

Simian adenoviruses of the invention can be generated using techniques known to those of skill in the art. They can be produced in a host cell line, i.e., any suitable cell line in which the virus is capable of replication. Replication defective viruses can be produced, e.g., in complementing cell lines which provide the factors missing from the viral vector that result in its impaired replication characteristics (such as E1).

Vectors of the invention are generated using techniques and sequences provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

### Excipients

Excipients of the invention can include buffers, salts, surfactants, sugars, organic compounds, chelating agents and proteins. Excipients of the invention act to stabilize the simian adenovirus while it is in an aqueous formulation.

“Buffer” refers to a substance capable of neutralizing both an acid and a base, thereby maintaining the pH of a solution. Suitable buffers of the invention include Tris, succinate, borate, Tris-maleate, lysine, histidine, glycine, glycylglycine, citrate, carbonate or combinations thereof.

“Aqueous” refers to water. An aqueous composition is one in which the solvent is water.

“Salt” refers to ionic compounds that result from the neutralization reaction of an acid and a base, composed of a related number of cations and anions such that the product is without net charge, for example sodium chloride. The component ions can be either inorganic or organic, and can be monoatomic or polyatomic.

“Amorphous sugar” refers to a sugar in which the constituent particles are arranged in a random manner.

“Chelating agent” refers to a chemical substance that reacts with metal ions to form a stable water soluble complex. Excipients of the invention can include a chelating agent selected from ethylenediaminetetraacetic acid (EDTA) and ethylene glycol-bis (beta-aminoethyl ether)-N,N,N′,N′-tetraacetic acid (EGTA).

“Surfactant” refers to a substance that reduces the surface tension of a liquid in which it is dissolved. Excipients of the invention can include a surfactant selected from polysorbate surfactants (e.g. polysorbate 80 and/or polysorbate 20), poloxamer surfactants (e.g. poloxamer 188), octoxinal surfactants, polidocanol surfactants, polyoxyl stearate surfactants, polyoxyl castor oil surfactants, N-octyl-glucoside surfactants, macrogol 15 hydroxy stearate, and combinations thereof. In an embodiment, the surfactant is selected from poloxamer surfactants (e.g. poloxamer 188), polysorbate surfactants (e.g. polysorbate 80 and/or polysorbate 20), in particular polysorbate surfactants such as polysorbate 80. Surfactants can also be used to formulate tocopherols.

“Vitamin E,” i.e., tocopherol, refers to a series of chiral organic molecules that vary in their degree of methylation of the phenol moiety of the chromanol ring. Tocopherols act as lipid soluble anti-oxidants.

The term includes alpha, beta, gamma and delta tocopherols. Various tocopherol salts include tocopherol succinate, tocopherol acetate, tocopherol nicotinate and other esterified forms.

By “Vitamin E succinate” or “VES” is meant any alpha tocopherol succinate, including but not limited to 4-oxo-4-[[2,5,7,8-tetramethyl-2-(4,8,12-trimethyltridecyl)-3,4-dihydrochromen-6-yl]oxy]butanoic acid, D-alpha-tocopherol succinate, semisynthetic D-alpha-tocopherol succinate, alpha tocopherol acid succinate, RRR-alpha-tocopherol hydrogen succinate, D-alpha-tocopherol succinate, (+)-alpha-tocopherols, (+)-delta tocopherols, tocopherol hemisuccinate and tocopheryl acid succinate. Typically, the empirical formula will be C₃₃H₅₄O₅ (Hill notation). VES can be solubilized in ethanol, which may be present in residual amounts in a buffer comprising VES.

By “recombinant human serum albumin,” “rHSA” or “human albumin” is meant a protein encoded by the human ALB gene and produced by recombination methods known in the art.

TRAVASOL is a solution of essential and nonessential amino acids comprising leucine, isoleucine, lysine, valine, phenylalanine, histidine, threonine, methionine, tryptophan, alanine, arginine, glycine, proline, serine and tyrosine with acetate and chloride anions at pH 5.0-7.0.

### Stability and Infectivity

Adenovirus is stable when frozen but, unless formulated appropriately, rapidly degrades at warmer temperatures. Degradation can be caused by factors such as, but not limited to, heat; oxidation, e.g., due to light, peroxide or metals; shear stress; the impact of an air interface on a liquid formulation; freeze thaw cycles; or combinations of these factors. The pathways by which adenoviruses undergo degradation include capsid disruption, aggregation, oxidation and deamidation.

“Stability” refers to resistance to degradation. Adenoviral stability can be measured by the integrity of the viral capsid. For example, the PICOGREEN assay utilizes a fluorescent nucleic acid stain that is selective for double stranded DNA. HPLC can quantify intact viral particles; the percentage of released DNA is based on a regression curve comprising a freshly thawed control group and a completely degraded control. Quantitative PCR can be used to quantify viral DNA (gE/ml=genome equivalents per ml.)

“Infectivity” refers to the ability of a vector to enter a susceptible host, i.e. a cell, and deliver its genetic material for expression by the host. Infectivity can be determined by measuring the entry of viral particles into cells, for example by immunostaining a viral protein. Infectivity assays are known in the art and include “cell culture infectious dose 50% (CCID₅₀)”, single “infectious unit (IFU)” plaque assays and hexon protein immunostaining. Infectivity can also be determined by measuring the proportion of cells that express a transgene, e.g., by FACS analysis or other suitable method. For example, any suitably expressed transgene, e.g., green fluorescent protein (“GFP”), Protein M or an antigenic polypeptide can be used as an infectivity marker whereby the number of cells expressing the transgene after incubation with the vector is measured.

The CCID₅₀ is the infectious dose that will infect 50% of the cells challenged with the defined inoculum and can be used to measure viral amplification and cell re-infection. Measuring the CCID₅₀ measures viral entry into the cells and viral replication. It does not measure the number of virus particles. The read-out for the CCID₅₀ is immunostaining of the hexon protein, determined by microscopy. The quantification is based on the amount of virus required to infect 50% of the cultured cells and can be expressed as log CCID₅₀/ml.

Infectious units (“IFU”) provide a measure of the number of infectious virus particles, e.g., per ml or per dose, and provide a measure of viral entry in to the cell and replication. The IFU is a plaque-based assay, the read-out is immunostaining of the hexon protein, determined by microscopy and can be expressed as infectious units per milliliter (IFU/ml). The quantification is based on the number of infective particles, with each plaque representing one infective particle.

Infectivity by hexon or a transgene refers to the ability to infect cells at a given time point and can be used to measure viral replication. The read-out can be, e.g., immunostaining of the transgene or the hexon protein, e.g., determined by FACS analysis, wherein the quantification is based on the number of positive cells.

### Embodiments of the Invention

In specific embodiments, the adenoviral vector is derived from a simian adenovirus. For example, the simian adenovirus may be an adenovirus from a New World monkey, an Old World monkey or an ape. It may be an adenovirus from a chimpanzee, a bonobo, a gorilla, an orangutan or a macaque. Chimpanzee adenoviruses of the invention include, but are not limited to ChAd3, ChAd63, ChAd83, ChAd155, ChAd157, ChAdOx1 and ChAdOx2. Examples of such strains are described in WO03/000283, WO2010/086189 and WO 2016/198621. Bonobo adenoviruses of the invention include but are not limited to PanAd1, PanAd2 or PanAd3, Pan 5, Pan 6, Pan 7 (also referred to as C7) and Pan 9. Bonobo vectors can be found, for example, in WO2005/071093 and WO2010/086189. Gorilla adenoviruses of the invention include but are not limited to GADNOU19, GADNOU20, GADNOU21, GADNOU25, GADNOU26, GADNOU27, GADNOU28, GADNOU29, GADNOU30 and GADNOU31. Gorilla vectors can be found, for example, in WO2013/52799, WO2013/52811, WO2013/52832 and WO2019/008111.

Compositions of the invention comprise a buffer selected from Tris, succinate, borate, Tris-maleate, lysine, histidine, glycine, glycylglycine, citrate, carbonate or combinations thereof. In one embodiment, the buffer is Tris, succinate or borate. In a further embodiment, the buffer is Tris. The buffer can be present in an amount of at least 1 mM, at least 2.5 mM or at least 5 mM. The buffer can be present in an amount of less than 25 mM, less than 20 mM or less than 15 mM. For example, the buffer can be present in an amount of 1 to 25 mM, 2.5 to 20 mM or 5 to 15 mM. In an embodiment, the buffer is present in an amount of about 10 mM.

In an embodiment, compositions of the invention also comprise histidine in an amount of up to about 20 mM, for example at a concentration of about 10 mM.

In an embodiment, compositions of the invention also comprise NaCl in an amount of up to about 50 mM, for example, at a concentration of about 5 mM.

In an embodiment, compositions of the invention also comprise bivalent metal ions, such as Mg²⁺ or Ca²⁺ or bivalent metal ions in the form of a salt, such as MgCl₂, CaCl₂) or MgSO₄. In an embodiment the bivalent metal ion is Mg²⁺. The bivalent metal ions can be present in an amount of about 0.1 to 10 mM, about 0.5 to 5 mM or about 0.5 to 2.0 mM. In an embodiment, the bivalent metal ion is MgCl₂ and is present in an amount of about 1 mM. In an embodiment, compositions of the invention contain no exogenous bivalent metal ions, i.e., none have been included in the formulation.

In an embodiment, compositions of the invention also comprise a poloxamer surfactant or a polysorbate surfactant. In an embodiment, the poloxamer surfactant is poloxamer 188. In an embodiment, the polysorbate surfactant is polysorbate 80 and/or polysorbate 20. The surfactant can be present in an amount of about 0.01 to 0.05% (w/v), such as about 0.02%, 0.025%, 0.03%, 0.035%, 0.04% or 0.045%. In an embodiment, the surfactant is polysorbate 80 and is present in an amount of about 0.020 to 0.030%, or about 0.025% (w/v).

In an embodiment, compositions of the invention also comprise an alpha tocopherol succinate. In an embodiment, the alpha tocopherol succinate is Vitamin E succinate. The alpha tocopherol succinate can be present in an amount up to about 0.5 mM, such as about 0.01 to 0.25 mM, such as 0.05 mM, 0.10 mM, 0.15 mM or 0.20 mM. In an embodiment, the alpha tocopherol succinate is Vitamin E succinate and is present in an amount of about 0.05 mM.

In an embodiment, compositions of the invention also comprise recombinant human serum albumin in an amount up to about 1% (w/v), for example in an amount of about 0.01%, 0.025%, 0.05%. 0.075%, 0.1%, 0.2%, 0.3%, 0.4%, 0.5%, 0.6%, 0.7%, 0.8% or about 0.9%.

In an embodiment, compositions of the invention comprise at least one amorphous sugar, such as sucrose, trehalose, mannose, mannitol, raffinose, lactitol, sorbitol and lactobionic acid, glucose, maltulose, iso-maltulose, lactulose, maltose, lactose, isomaltose, maltitol, palatinit, stachyose, melezitose, dextran, or a combination thereof. In an embodiment, the amorphous sugar is trehalose, sucrose or combination of sucrose and trehalose. In an embodiment, the amorphous sugar is sucrose. In an embodiment, the amorphous sugar is a combination of sucrose and trehalose. In an embodiment, the amorphous sugar is trehalose.

In an embodiment, the amorphous sugar is present in an amount of about 0% to 30% (w/v). In an embodiment, the amorphous sugar is sucrose in an amount up to about 30% (w/v). In an embodiment, sucrose is present in an amount of about 0 to 10% or 0 to 5% (w/v), for example in an amount of about 2%. In another embodiment, sucrose is present in an amount of about 10 to 30% (w/v), for example in an amount of about 16%.

In an embodiment, the amorphous sugar is trehalose in an amount up to about 30% (w/v). In an embodiment, trehalose is present in an amount of about 10 to 20% (w/v), for example in an amount of about 15%.

In an embodiment, the amorphous sugar is a combination of sucrose and trehalose in an amount of about 0 to 10% sucrose (w/v) and about 10 to 30% trehalose (w/v); in an amount of about 0 to 5% sucrose and 10 to 20% trehalose, for example in an amount of about 2% sucrose and 15% trehalose.

In a particular embodiment, compositions of the invention comprise about 1 to 25 mM Tris pH 6-10, about 0 to 15 mM histidine, about 0 to 50 mM NaCl, about 0.1 to 10 mM MgCl₂, about 0.01 to 0.05% polysorbate 80 (w/v), about 0.001 to 0.5 mM Vitamin E succinate, about 0.01 to 1% (w/v) recombinant human serum albumin and about 0 to 30% (w/v) trehalose. In another particular embodiment, compositions of the invention comprise about 1 to 25 mM Tris pH 6-10, about 0 to 15 mM histidine, about 0 to 50 mM NaCl, about 0.01 to 0.05% polysorbate 80 (w/v), about 0.001 to 0.5 mM Vitamin E succinate, about 0.01 to 1% (w/v) recombinant human serum albumin and about 0 to 30% (w/v) trehalose.

In a particular embodiment, compositions of the invention comprise about 1 to 25 mM Tris pH 6-10, about 0 to 15 mM histidine, about 0 to 50 mM NaCl, about 0.1 to 10 mM MgCl₂, about 0.01 to 0.05% (w/v) polysorbate 80, about 0.001 to 0.5 mM Vitamin E succinate, about 0.01 to 1% (w/v) recombinant human serum albumin, about 0 to 10% (w/v) sucrose and about 5 to 25% (w/v) trehalose. In another particular embodiment, compositions of the invention comprise about 1 to 25 mM Tris pH 6-10, about 0 to 15 mM histidine, about 0 to 50 mM NaCl, about 0.01 to 0.05% (w/v) polysorbate 80, about 0.001 to 0.5 mM Vitamin E succinate, about 0.01 to 1% (w/v) recombinant human serum albumin, about 0 to 10% (w/v) sucrose and about 5 to 25% (w/v) trehalose.

In a more particular embodiment, compositions of the invention comprise about 2.5 to 20 mM Tris pH 7.5 to 9.5, about 5 to 15 mM histidine, about 0 to 10 mM NaCl, about 0.5 to 5 mM MgCl₂, about 0.01 to 0.05% (w/v) polysorbate 80 w/v, about 0.01 to 0.25 mM Vitamin E succinate, about 0 to 1% w(w/v) recombinant human serum albumin and about 5 to 25% (w/v) trehalose. In another particular embodiment, compositions of the invention comprise about 2.5 to 20 mM Tris pH 7.5 to 9.5, about 5 to 15 mM histidine, about 0 to 10 mM NaCl, about 0.01 to 0.05% (w/v) polysorbate 80 w/v, about 0.01 to 0.25 mM Vitamin E succinate, about 0 to 1% w(w/v) recombinant human serum albumin and about 5 to 25% (w/v) trehalose.

In another more particular embodiment, compositions of the invention comprise about 2.5 to 20 mM Tris pH 7.5 to 9.5, about 5 to 15 mM histidine, about 0 to 10 mM NaCl, about 0.5 to 5 mM MgCl₂, about 0.01 to 0.05% (w/v) polysorbate 80, about 0.01 to 0.25 mM Vitamin E succinate, about 0 to 1% (w/v) recombinant human serum albumin and about 10 to 20% (w/v) sucrose. In another particular embodiment, compositions of the invention comprise about 2.5 to 20 mM Tris pH 7.5 to 9.5, about 5 to 15 mM histidine, about 0 to 10 mM NaCl, about 0.01 to 0.05% (w/v) polysorbate 80, about 0.01 to 0.25 mM Vitamin E succinate, about 0 to 1% (w/v) recombinant human serum albumin and about 10 to 20% (w/v) sucrose.

In a further more particular embodiment, compositions of the invention comprise about 5 to 15 mM Tris pH 7.5 to 9.5, about 8 to 12 mM histidine, about 0.5 to 2.5 mM NaCl, about 0.5 to 5 mM MgCl₂, about 0.015 to 0.035% (w/v) polysorbate 80, about 0.025 to 0.1 mM Vitamin E succinate, about 0.1 to 0.5% (w/v) recombinant human serum albumin and about 0 to 5% (w/v) sucrose. In another particular embodiment, compositions of the invention comprise about 5 to 15 mM Tris pH 7.5 to 9.5, about 8 to 12 mM histidine, about 0.5 to 2.5 mM NaCl, about 0.015 to 0.035% (w/v) polysorbate 80, about 0.025 to 0.1 mM Vitamin E succinate, about 0.1 to 0.5% (w/v) recombinant human serum albumin and about 0 to 5% (w/v) sucrose.

In a yet further more particular embodiment, compositions of the invention comprise about 5 to 15 mM Tris pH 7.5 to 9.5, about 8 to 12 mM histidine, about 0.5 to 2.5 mM NaCl, about 0.5 to 5 mM MgCl₂, about 0.015 to 0.035% (w/v) polysorbate 80, about 0.025 to 0.1 mM Vitamin E succinate, about 0.1 to 0.5% (w/v) recombinant human serum albumin, about 0 to 5% (w/v) sucrose and about 10 to 20% (w/v) trehalose. In another particular embodiment, compositions of the invention comprise about 5 to 15 mM Tris pH 7.5 to 9.5, about 8 to 12 mM histidine, about 0.5 to 2.5 mM NaCl, about 0.015 to 0.035% (w/v) polysorbate 80, about 0.025 to 0.1 mM Vitamin E succinate, about 0.1 to 0.5% (w/v) recombinant human serum albumin, about 0 to 5% (w/v) sucrose and about 10 to 20% (w/v) trehalose.

In a yet further more particular embodiment, compositions of the invention comprise about 5 to 15 mM Tris pH 7.5 to 9.5, about 8 to 12 mM histidine, about 0.5 to 2.5 mM NaCl, about 0.5 to 5 mM MgCl₂, about 0.015 to 0.035% (w/v) polysorbate 80, about 0.025 to 0.1 mM Vitamin E succinate, about 0.1 to 0.5% (w/v) recombinant human serum albumin and about 10 to 20% (w/v) sucrose. In another particular embodiment, compositions of the invention comprise about 5 to 15 mM Tris pH 7.5 to 9.5, about 8 to 12 mM histidine, about 0.5 to 2.5 mM NaCl, about 0.015 to 0.035% (w/v) polysorbate 80, about 0.025 to 0.1 mM Vitamin E succinate, about 0.1 to 0.5% (w/v) recombinant human serum albumin and about 10 to 20% (w/v) sucrose.

In a most particular embodiment, compositions of the invention comprise about 10 mM Tris pH 8.5, about 10 mM histidine, about 5 mM NaCl, about 1 mM MgCl₂, about 0.024% (w/v) polysorbate 80, about 0.05 mM Vitamin E succinate, about 0.1% (w/v) recombinant human serum albumin and about 16% (w/v) trehalose. In another particular embodiment, compositions of the invention comprise about 10 mM Tris pH 8.5, about 10 mM histidine, about 5 mM NaCl, about 0.024% (w/v) polysorbate 80, about 0.05 mM Vitamin E succinate, about 0.1% (w/v) recombinant human serum albumin and about 16% (w/v) trehalose.

In another most particular embodiment, compositions of the invention comprise about 10 mM Tris pH 8.5, about 10 mM histidine, about 5 mM NaCl, about 1 mM MgCl₂, about 0.024% w(w/v) polysorbate 80, about 0.05 mM Vitamin E succinate, about 0.1% (w/v) recombinant human serum albumin, about 2% (w/v) sucrose and about 15% (w/v) trehalose. In another particular embodiment, compositions of the invention comprise about 10 mM Tris pH 8.5, about 10 mM histidine, about 5 mM NaCl, about 0.024% w(w/v) polysorbate 80, about 0.05 mM Vitamin E succinate, about 0.1% (w/v) recombinant human serum albumin, about 2% (w/v) sucrose and about 15% (w/v) trehalose.

In a further most particular embodiment, compositions of the invention comprise about 10 mM Tris pH 8.5, about 10 mM histidine, about 5 mM NaCl, about 1 mM MgCl₂, about 0.024% (w/v) polysorbate 80, about 0.05 mM Vitamin E succinate, about 0.1% (w/v) recombinant human serum albumin, and about 16% (w/v) sucrose. In another particular embodiment, compositions of the invention comprise about 10 mM Tris pH 8.5, about 10 mM histidine, about 5 mM NaCl, about 0.024% (w/v) polysorbate 80, about 0.05 mM Vitamin E succinate, about 0.1% (w/v) recombinant human serum albumin, and about 16% (w/v) sucrose.

In an embodiment, the invention provides a method of making an aqueous liquid composition described herein by producing a simian adenovirus in a host cell and combining the adenovirus with one or more excipients, wherein the adenovirus is stable at +2-+8° C. for at least two years.

In another embodiment, the invention provides a method of making an aqueous liquid composition described herein by producing a simian adenovirus in a host cell and combining the adenovirus with one or more excipients, wherein the adenovirus is stable at 25° C. for at least 30 days.

In a further embodiment, the invention provides a method of making an aqueous liquid composition described herein by producing a simian adenovirus in a host cell and combining the adenovirus with one or more excipients, wherein the adenovirus is stable at 30° C. for at least seven days.

The aqueous compositions of the invention may be contained in a glass vial, which may be either siliconized or non-siliconized. They may be contained in prefilled syringes, plastic containers with or without a blow-fill-seal, microneedle devices for intra-dermal administration and other containers used for containing viruses.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise.

Numerical limitations given with respect to concentrations or levels of a substance, such as solution component concentrations or ratios thereof, and reaction conditions such as temperatures, pressures and cycle times are intended to be approximate. The term “about” used herein is intended to mean the amount±10%.

The term “comprises” means “includes.” Thus, unless the context requires otherwise, the word “comprises,” and variations such as “comprise” and “comprising,” are understood to imply the inclusion of a stated compound or composition (e.g., nucleic acid, polypeptide, antigen) or step, or group of compounds or steps, but no to the exclusion of other compounds, compositions, steps or groups thereof. The abbreviation, “e.g.” is derived from the Latin exempli gratia, and is used herein to indicate a non-limiting example. Thus, the abbreviation “e.g.” is synonymous with the term “for example.”

The term “substantially” does not exclude “completely.” For example, a composition that is substantially free from X may be completely free from X.

The present invention will now be further described by means of the following non-limiting examples.

### EXAMPLES

### Example 1: Factorial Testing of the Effects of Excipients on Stability

ChAd155-RSV (WO 2016/198599) was formulated at a concentration of 1×10¹¹ virus particle units per milliliter (“pu/ml”) in 10 mM Tris pH 8.5, 25 mM NaCl, 8% sucrose (w/v), 0.02% polysorbate 80 (w/v) and 1 mM MgCl₂, then tested for stability after being exposed to temperatures of 30° C. for three days or seven days. Five excipients were tested in a 2⁵⁻¹ fractional factorial design study having five factors (the excipients) with 16 runs in the corners and four center points. The factors were (1) MgSO₄ at a concentration of 15 mM or 30 mM; (2) ethanol at a concentration of 1.54% or 3.07%; (3) TRAVASOL at a concentration of 1 mM or 4 mM; (4) rHSA at a concentration of 0.05% or 0.5%; and (5) Vitamin E succinate (VES) at a concentration of 1 mM or 2 mM. Stability was determined by measuring viral particles by analytical HPLC. The ability of the virus to retain infectivity was measured as the number of cells expressing the adenovirus hexon capsid protein after incubation with the virus and the comparisons are shown in the tables below.

| Run | MgSO₄ | EtOH | TRAVASOL | rHSA | VES |
|---|---|---|---|---|---|
| 1 | 1 | −1 | 1 | −1 | 1 |
| 2 | 0 | 0 | 0 | 0 | 0 |
| 3 | 1 | −1 | −1 | −1 | −1 |
| 4 | 1 | −1 | −1 | 1 | 1 |
| 5 | 1 | 1 | 1 | 1 | 1 |
| 6 | −1 | 1 | 1 | −1 | 1 |
| 7 | 0 | 0 | 0 | 0 | 0 |
| 8 | −1 | 1 | −1 | −1 | −1 |
| 9 | 1 | 1 | 1 | −1 | −1 |
| 10 | −1 | −1 | −1 | 1 | −1 |
| 11 | −1 | −1 | 1 | −1 | −1 |
| 12 | −1 | −1 | 1 | 1 | 1 |
| 13 | 0 | 0 | 0 | 0 | 0 |
| 14 | 1 | 1 | −1 | −1 | 1 |
| 15 | −1 | −1 | −1 | −1 | 1 |
| 16 | −1 | 1 | 1 | 1 | −1 |
| 17 | 1 | 1 | −1 | 1 | −1 |
| 18 | 1 | −1 | 1 | 1 | −1 |
| 19 | −1 | 1 | −1 | 1 | 1 |
| 20 | 0 | 0 | 0 | 0 | 0 |
| Value | MgSO₄ | EtOH | TRAVASOL | rHSA | VES |
| 0 | 0 mM | 0% | 0 mM | 0% | 0 mM |
| 1 | 30 mM | 3.07% | 4 mM | 0.5% | 2 mM |
| −1 | 15 mM | 1.54% | 1 mM | 0.05% | 1 mM |

PICOGREEN analysis showed that VES contributed about 30% of the impact and had a positive effect on the stability of the virus. Travasol contributed about 18% and had a negative impact. The other factors tested had a negligible impact in this analysis, both alone and in combination with other factors.

Considering both stability, as measured by PICOGREEN and analytical HPLC; and infectivity, as measured by hexon protein, the global ranking of the five excipients was VES>rHSA>ethanol>MgSO₄>TRAVASOL. VES had a positive impact on the adenovirus' ability to withstand heat treatment. VES accounted for 50% of the variability contribution to heat stability and 20% of the variability contribution to infectivity. Recombinant HSA increased the thermostability of the virus, while VES both increased the thermostability and prevented metal-catalyzed oxidation following light exposure. TRAVASOL and MgSO₄ both had a negative impact on infectivity, with the latter inducing a 17% loss in infectivity between days three and seven at 30° C.

The inventors found that rHSA prevented adenovirus from aggregating in solution. Simian adenovirus with a respiratory syncytial virus (RSV) transgene was compared in the presence and absence of rHSA. The results of gel electrophoresis experiments demonstrated that rHSA substantially reduced the formation of high molecular weight adenoviral multimers. Recombinant HSA also has antioxidant effects.

### Example 2: Effect of Sugar Composition, Osmolality, VES and rHSA on Stability and Infectivity

ChAd155-RSV was formulated at a concentration of 1×10¹¹ pu/ml comprising a 10% overage in Tris pH 8.5, NaCl, polysorbate 80 (w/v), histidine, MgCl₂, trehalose, sucrose, rHSA and VES at concentrations shown in the table below for the first twelve compositions. Compositions 13-15 were formulated at 2× concentration to test the concept of a syrup and were then diluted prior to analysis.

| ¶ | ¶ | ¶ | ¶ | ¶ | ¶ | | | | ¶ | | |
|---|---|---|---|---|---|---|---|---|---|---|---|
| ¶ | ¶ | ¶ | ¶ | ¶ | ¶ | ¶ | ¶ | ¶ | rHSA¶ | ¶ | ¶ |
| #¤ | Adenovirus¤ | Tris•pH [symbol] 8.5•(mM)¤ | Histidine•(mM)¤ | NaCl•(nM)¤ | MgCl₂•(nM)¤ | Polysorbate•80•(%•w/v)¤ | Trehalose•(%•w/v)¤ | Sucrose•(%•w/v)¤ | (%•w/v)¤ | VES¶ (mM)¤ | Osmolality•(mOsm)¤ |
| 1¤ | 1.1 × | 10¤ | 10¤ | 5¤ | 1¤ | 0.020¤ | 7 (low)¤ | 2¤ | 0¤ | 0¤ | 325¤ |
| | 10¹¹¤ | | | | | | | | | | |
| 2¤ | 1.1 × | 10¤ | 10¤ | 5¤ | 1¤ | 0.020¤ | 15 (mid)¤ | 2¤ | 0¤ | 0¤ | 616¤ |
| | 10¹¹¤ | | | | | | | | | | |
| 3¤ | 1.1 × | 10¤ | 10¤ | 5¤ | 1¤ | 0.020¤ | 23 (high)¤ | 2¤ | 0¤ | 0¤ | 584¤ |
| | 10¹¹¤ | | | | | | | | | | |
| 4¤ | 1.1 × | 10¤ | 10¤ | 5¤ | 1¤ | 0.024¤ | 7 (low)¤ | 2¤ | 0¤ | 0.5¤ | 333¤ |
| | 10¹¹¤ | | | | | | | | | | |
| 5¤ | 1.1 × | 10¤ | 10¤ | 5¤ | 1¤ | 0.024¤ | 15 (mid)¤ | 2¤ | 0¤ | 0.5¤ | 640¤ |
| | 10¹¹¤ | | | | | | | | | | |
| 6¤ | 1.1 × | 10¤ | 10¤ | 5¤ | 1¤ | 0.024¤ | 23 (high)¤ | 2¤ | 0¤ | 0.5¤ | 1013¤ |
| | 10¹¹¤ | | | | | | | | | | |
| 7¤ | 1.1 × | 10¤ | 10¤ | 5¤ | 1¤ | 0.024¤ | 7 (low)¤ | 2¤ | 0.1¤ | 0.5¤ | 337¤ |
| | 10¹¹¤ | | | | | | | | | | |
| 8¤ | 1.1 × | 10¤ | 10¤ | 5¤ | 1¤ | 0.024¤ | 15 (mid)¤ | 2¤ | 0.1¤ | 0.5¤ | 645¤ |
| | 10¹¹¤ | | | | | | | | | | |
| 9¤ | 1.1 × | 10¤ | 10¤ | 5¤ | 1¤ | 0.024¤ | 23 (high)¤ | 2¤ | 0.1¤ | 0.5¤ | 1020¤ |
| | 10¹¹¤ | | | | | | | | | | |
| 10¤ | 1.1 × | 10¤ | 0¤ | 25¤ | 1¤ | 0.020¤ | 0¤ | 8¤ | 0¤ | 0¤ | 337¤ |
| | 10¹¹¤ | | | | | | | | | | |
| 11¤ | 1.1 × | 10¤ | 0¤ | 25¤ | 1¤ | 0.020¤ | 0¤ | 16¤ | 0¤ | 0¤ | 661¤ |
| | 10¹¹¤ | | | | | | | | | | |
| 12¤ | 1.1 × | 10¤ | 0¤ | 25¤ | 1¤ | 0.020¤ | 0¤ | 25¤ | 0¤ | 0¤ | 1151¤ |
| | 10¹¹¤ | | | | | | | | | | |
| 13¤ | 2.2 × | 20¤ | 20¤ | 7¤ | 2¤ | 0.040¤ | 35¤ | 2.2¤ | 0¤ | 0¤ | >1000¤ |
| | 10¹¹¤ | | | | | | | | | | |
| 14¤ | 2.2 × | 20¤ | 20¤ | 7¤ | 2¤ | 0.040¤ | 0¤ | 35¤ | 0¤ | 0¤ | >1000¤ |
| | 10¹¹¤ | | | | | | | | | | |
| 15¤ | 2.2 × | 20¤ | 20¤ | 7¤ | 2¤ | 0.040¤ | 0¤ | 48¤ | 0¤ | 0¤ | >1000¤ |
| | 10¹¹¤ | | | | | | | | | | |

[symbol]

indicates data missing or illegible when filed

Each of the above compositions was tested for stability after being exposed to temperatures of 4° C. for three months or 30° C. for one month to examine the effects of high osmolality and sugar composition on stability and infectivity. Osmolality had little or no effect on either stability or infectivity.

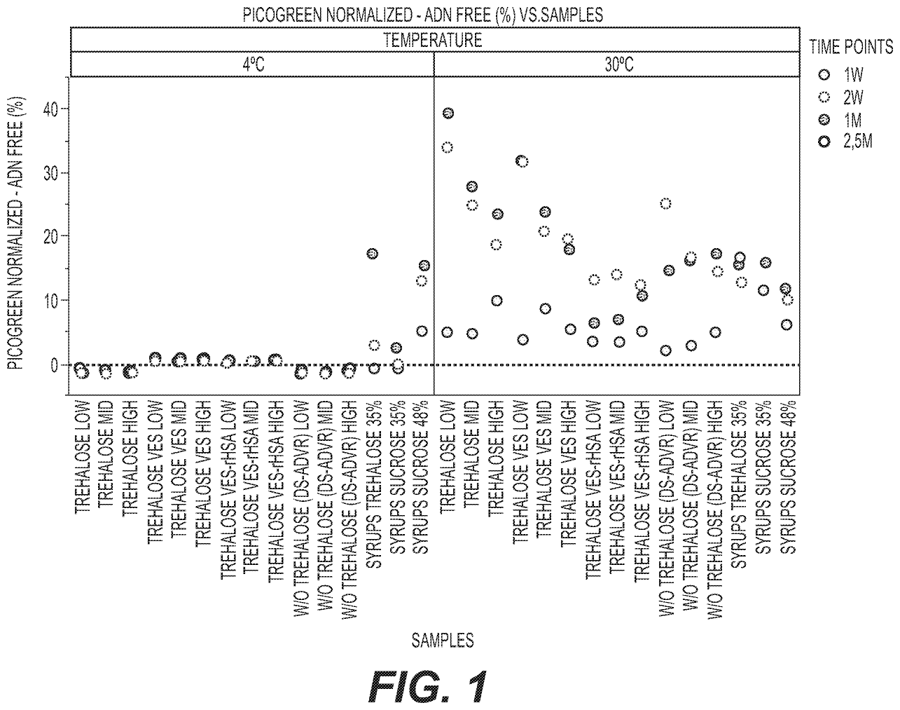

As shown in FIG. 1, most of the virus compositions were stable for at least two and a half months at 4° C. (left panel) and one week at 30° C. (right panel). After three months at 4° C. compositions 1-12 remained stable. After two weeks or one month at 30° C. (right panel dotted-outlined dots and dotted-filled dots, respectively), the addition of VES to the compositions containing either low, medium or high concentrations of trehalose demonstrated increased viral stability compared to the composition in the absence of VES, as measured by a decrease in the amount of free DNA. The same positive effect on stability was observed with the addition of both VES and rHSA to compositions containing low, medium or high concentrations of trehalose. Replacing trehalose with sucrose reduced the beneficial effect of VES and VES+rHSA, as did doubling the sugar concentration to produce a syrup.

### Example 3: Effect of Trehalose, Sucrose, VES and rHSA on Stability at 4° C. or 25° C.

ChAd155-RSV was formulated as shown in the table below. Each formulation was tested for stability by analytical HPLC after being exposed to temperatures of 4° C. or 25° C. for three weeks to examine the effects of trehalose, sucrose, VES and rHSA on stability.

| | | | | | | Polysorbate | Trehalose | Sucrose | rHSA | |
|---|---|---|---|---|---|---|---|---|---|---|
| | Adenovirus | Tris | Histidine | NaCl | MgCl2 | (% | (% | (% | (% | VES |
| | (pu/ml) | (mM) | (mM) | (mM) | (mM) | w/v) | w/v) | w/v) | w/v) | (mM) |
| 1 | 1.1 × 10¹¹ | 10 | 10 | 5 | 1 | 0.024 | 7 | 2 | 0 | 0 |
| 2 | 1.1 × 10¹¹ | 10 | 10 | 5 | 1 | 0.024 | 7 | 2 | 0 | 0.05 |
| 3 | 1.1 × 10¹¹ | 10 | 0 | 5 | 1 | 0.024 | 0 | 8 | 0 | 0 |
| 4 | 1.1 × 10¹¹ | 10 | 0 | 5 | 1 | 0.024 | 0 | 8 | 0 | 0.05 |
| 5 | 1.1 × 10¹¹ | 10 | 10 | 5 | 1 | 0.024 | 0 | 8 | 0.1 | 0.05 |
| 6 | 1.1 × 10¹¹ | 10 | 10 | 25 | 1 | 0.024 | 0 | 8 | 0 | 0 |
| 7 | 1.1 × 10¹¹ | 10 | 10 | 25 | 1 | 0.024 | 0 | 8 | 0.1 | 0.05 |

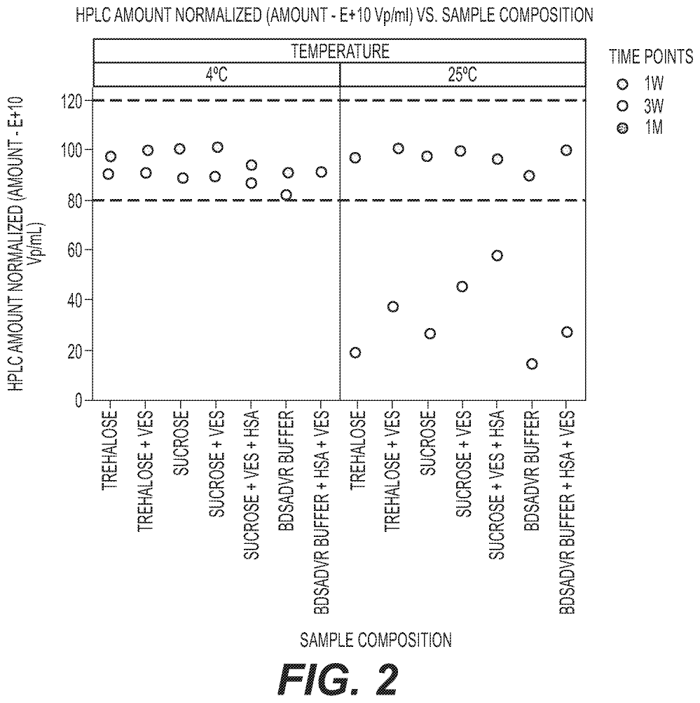

The virus was maintained at 4° C. for three weeks (left panel) and at 25° C. for at least one week (right panel) in each of the tested formulations (FIG. 2). After one week at 25° C., no degradation was observed. After three weeks at 25° C., some degradation began to occur and the differential effects of the formulation components were able to be observed. VES increased viral stability when included in either sucrose-based or trehalose-based formulations. The addition of rHSA further increased stability, both in the presence and absence of added sugar.

### Example 4: Effect of Trehalose, Sucrose, VES and rHSA on Stability and Infectivity at 4° C.

ChAd155-RSV was formulated in 10 mM Tris pH 8.5, 10 mM histidine, 5 mM NaCl, 1 mM MgCl₂ and 0.024% polysorbate 80 (w/v), with the addition of trehalose, sucrose, VES and rHSA as shown in the table below at a viral concentration of 1.1×10¹¹ pu/ml or 1.1×10¹⁰ pu/ml, taking into account a 10% overage. Stability was determined at 4° C. over the course of six months by sampling at days 0, 42, 91, 145 and 186 by HPLC in two separate HPLC runs. Infectivity was measured in the presence of sucrose and VES by an infectious unit plaque assay.

| Formulation: | | | | | |
|---|---|---|---|---|---|
| 10 mM Tris pH 8.5, | | | | | |
| 10 mM histidine, | | | Stability | Stability | |
| 5 mM NaCl, 1 mM | | | Measured | Measured | Infectivity |
| MgCl₂, 0.024% | Adenoviral | | by HPLC | by HPLC | Measured by |
| polysorbate 80 (w/v) | Concentration | Timepoint | (pu/ml) | (pu/ml) | IU (average) |
| with the addition of: | (pu/ml) | (days) | Run 1 | Run 2 | (IU/ml) |
| 15% trehalose | 1 × 10¹¹ | 0 | 1.15 × 10¹¹ | 1.15 × 10¹¹ | ND |
| 2% sucrose | | 42 | 1.07 × 10¹¹ | 1.05 × 10¹¹ | |
| 0.05 mM VES | | 91 | 1.02 × 10¹¹ | 1.00 × 10¹¹ | |
| 0.1% rHSA (w/v) | | 145 | 0.98 × 10¹¹ | 0.96 × 10¹¹ | |
| | | 186 | 0.90 × 10¹¹ | 0.88 × 10¹¹ | |
| 16% sucrose | l × 10¹¹ | 0 | 1.19 × 10¹¹ | 1.10 × 10¹¹ | 9.39 × 10¹⁰ ± 0.14 |
| 0.05 mM VES | | 42 | 1.15 × 10¹¹ | 1.05 × 10¹¹ | 9.38 × 10¹⁰ ± 0.13 |
| | | 91 | 1.14 × 10¹¹ | 1.04 × 10¹¹ | 9.38 × 10¹⁰ ± 0.14 |
| | | 145 | 1.12 × 10¹¹ | 1.03 × 10¹¹ | 9.33 × 10¹⁰ ± 0.22 |
| | | 186 | 1.10 × 10¹¹ | 1.01 × 10¹¹ | 9.26 × 10¹⁰ ± 0.16 |
| 15% trehalose | 1 × 10¹⁰ | 0 | 1.08 × 10¹⁰ | 0.95 × 10¹⁰ | ND |
| 2% sucrose | | 42 | 1.06 × 10¹⁰ | 0.95 × 10¹⁰ | |
| 0.05 mM VES | | 91 | 1.07 × 10¹⁰ | 0.96 × 10¹⁰ | |
| 0.1% rHSA | | 145 | 1.08 × 10¹⁰ | 0.93 × 10¹⁰ | |
| | | 186 | 1.08 × 10¹⁰ | 0.95 × 10¹⁰ | |

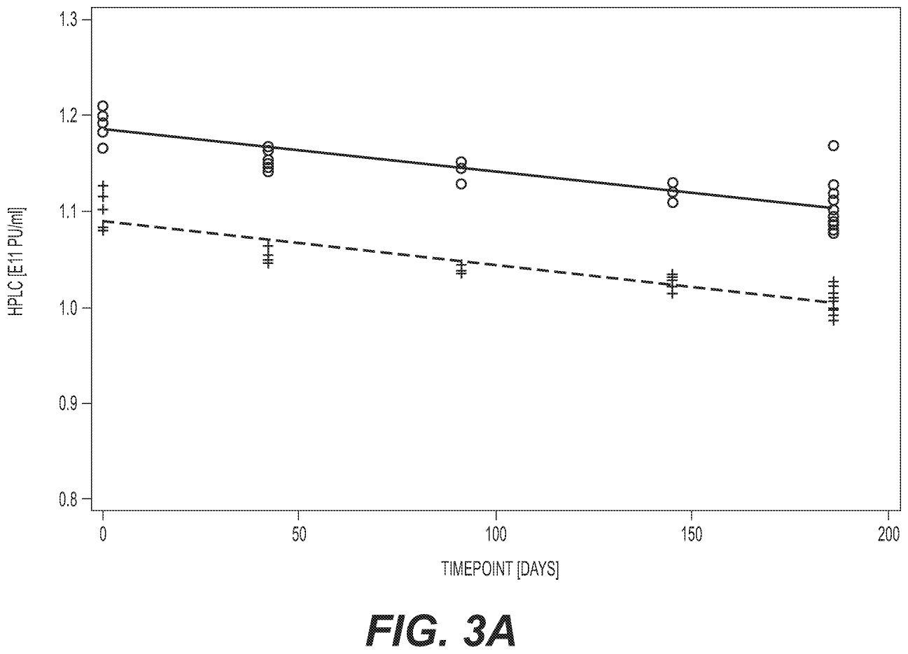

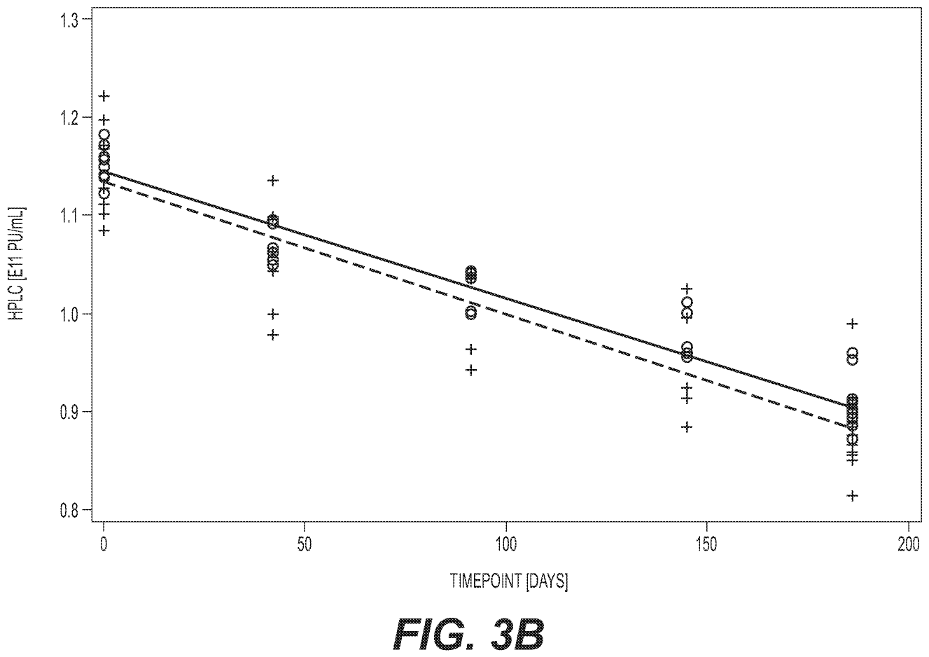

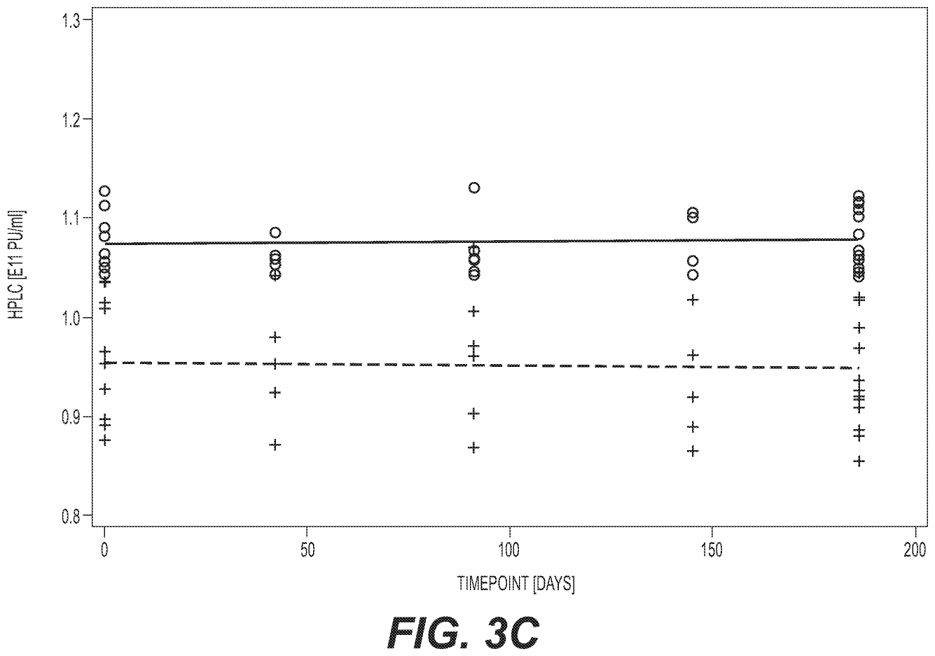

As shown in FIG. 3A, adenovirus formulated in 10 mM Tris pH 8.5, 5 mM NaCl, 0.024% polysorbate 80 (w/v), 1 mM MgCl₂, histidine 10 mM, 15% trehalose (w/v), 2% sucrose (w/v), 0.05 mM VES and 0.1% rHSA (w/v) at a concentration of 1.1×10¹¹ pu/ml observed over a period of 186 days at 4° C. showed a slow, steady decrease in stability. When extrapolated, these data project an average decline in total viral content of about 30% over two years and about 45% over three years.

Adenovirus formulated in 10 mM Tris pH 8.5, 5 mM NaCl, 0.024% polysorbate 80 (w/v), 1 mM MgCl₂, histidine 10 mM, 16% sucrose, and 0.05 mM VES at a concentration of 1.1×10¹¹ pu/ml observed over a period of 186 days 4° C. also showed a steady decrease in stability (FIG. 3B). When extrapolated, these data project an average decline in total viral content of about 85% over two years.

Adenovirus formulated in 10 mM Tris pH 8.5, 5 mM NaCl, 0.024% polysorbate 80 (w/v), 1 mM MgCl₂, histidine 10 mM, 15% trehalose (w/v), 2% sucrose (w/v), 0.05 mM VES and 0.1% rHSA (w/v) at a concentration of 1.1×10¹⁰ pu/ml observed over a period of 186 days 4° C. showed no significant loss of stability (Table and FIG. 3C).

### Example 5: Accelerated Stability Evaluations

The two experiments shown below further demonstrate that formulations of simian adenovirus comprising VES and rHSA are thermostable and retain their infectious properties.

### Experiment 1: 30 days at 4° C. or 25° C.

### Thermostability

Adenovirus was formulated at concentrations of 7.5×10¹⁰ pu/ml or 7.5×10⁹ pu/ml (including a 10% overage) in 10 mM Tris pH 8.5, 5 mM NaCl, 0.024% polysorbate 80 (w/v), 10 mM histidine and 1 mM MgCl₂, with the addition of either: (1) 16% sucrose (w/v)+0.05 mM VES; (2) 16% sucrose (w/v)+0.05% VES+0.1% rHSA (w/v); or (3) 15% trehalose (w/v)+2% sucrose (w/v)+0.05 mM VES+0.1% rHSA (w/v), as shown in the table below. The virus was kept at a temperature of either 4° C. or 25° C. for 30 days. Stability was evaluated by analytical HPLC, PICOGREEN, and quantitative PCR in the presence and absence of benzonase, which is both a DNA and an RNA nuclease.

| | Formulation | | | | | | |
|---|---|---|---|---|---|---|---|
| | 10 mM Tris pH | | | | | | |
| | 8.5, 10 mM | | | | | | |
| | histidine, 5 mM | | | | | | |
| | NaCl, 1 mM | | | | | | |
| | MgCl₂, 0.024% | | | | | | |
| | polysorbate | | | | | qPCR | |
| Adenovirus | 80 (w/v) | HPLC | | PICOGREEN | | (gE/ml) | |
| Concentration | with the | (pu/ml) | | (%) | | (+) or (−) benzonase | |
| (pu/ml) | addition of: | 4° C. | 25° C. | 4° C. | 25° C. | 4° C. | 25° C. |
| 7.5 × 10¹⁰ pu/ml | 16% sucrose | 7.08 × 10¹⁰ | 5.70 × 10¹⁰ | 2.2% | 2.8% | (+) 6.29 × 10¹⁰ | (+) 5.30 × 10¹⁰ |
| | 0.05 mM VES | | | | | (−) 6.69 × 10¹⁰ | (−) 5.71 × 10¹⁰ |
| | 16% sucrose | 7.40 × 10¹⁰ | 6.81 × 10¹⁰ | 2.6% | 3.8% | (+) 6.76 × 10¹⁰ | (+) 5.94 × 10¹⁰ |
| | 0.05 mM VES | | | | | (−) 6.83 × 10¹⁰ | (−) 7.02 × 10¹⁰ |
| | 0.1% rHSA | | | | | | |
| | 15% trehalose | 7.33 × 10¹⁰ | 5.97 × 10¹⁰ | 2.6% | 7.1% | (+) 6.53 × 10¹⁰ | (+) 4.75 × 10¹⁰ |
| | 0.05 mM VES | | | | | (−) 7.09 × 10¹⁰ | (−) 6.48 × 10¹⁰ |
| | 0.1% rHSA | | | | | | |
| 7.5 × 10⁹ pu/ml | 16% sucrose | 6.19 × 10⁹ | 6.15 × 10⁹ | ND | ND | ND | ND |
| | 0.05 mM VES | | | | | | |
| | 16% sucrose | 6.13 × 10⁹ | 6.14 × 10⁹ | ND | ND | ND | ND |
| | 0.05 mM VES | | | | | | |
| | 0.1% rHSA | | | | | | |
| | 15% trehalose | 6.37 × 10⁹ | 6.48 × 10⁹ | ND | ND | ND | ND |
| | 0.05 mM VES | | | | | | |
| | 0.1% rHSA | | | | | | |

These results demonstrate that all three compositions provide thermostable simian adenovirus formulations. As shown in the table above, as measured by analytical HPLC, no significant degradation was observed after 30 days at 25° C. in any of the three formulations tested at a viral concentration of 7.5×10⁹ pu/ml and there was no significant difference in stability among the formulations.

At a viral concentration of 7.5×10¹⁰ pu/ml, a viral particle loss of 8-19% was observed, with the greatest loss occurring in the sucrose+VES formulation. Loss occurring in the sucrose+VES+rHSA formulation was significantly less than observed with the other two formulations.

As measured by PICOGREEN, at a viral concentration of 7.5×10¹⁰ pu/ml, the normalized average of DNA released from the viral capsid was higher after 30 days at 25° C. than at 4° C. with all three formulations. As measured by qPCR, at a viral concentration of 7.5×10¹⁰ pu/ml, the number of genome equivalents per ml decreased in all three formulations after 30 days at 25° C. compared to 4° C., with the exception of the sucrose+VES+rHSA formulation assayed in the absence of benzonase. The least decrease was observed with sucrose+VES+rHSA in both the presence and absence of benzonase.

### Infectivity

Adenovirus was formulated at a concentration of 7.5×10¹⁰ pu/ml in 10 mM Tris pH 8.5, 5 mM NaCl, 0.024% polysorbate 80 (w/v), histidine 10 mM, 1 mM MgCl₂, with the addition of either: (1) 16% sucrose (w/v)+0.05 mM VES; (2) 16% sucrose (w/v)+0.05 mM VES+0.1% rHSA; or (3) 15% trehalose (w/v)+0.05 mM VES+0.1% rHSA. The virus was kept at a temperature of either 4° C. or 25° C. for 30 days.

| Adenovirus | | Infectivity | |
|---|---|---|---|
| Concentration | | (logl0(IU/ml)) | |
| (pu/ml) | Formulation | 4° C. | 25° C. |
| 6 × 10¹⁰ | 16% sucrose | 8.88 | 7.92 |
| | 0.05 mM VES | | |
| | 16% sucrose | 8.91 | 8.23 |
| | 0.05 mM VES | | |
| | 0.1% rHSA | | |
| | 15% trehalose | 8.89 | 7.96 |
| | 0.05 mM VES | | |
| | 0.1% rHSA | | |

Infectivity was determined by measuring infectious units by FACS analysis. At a viral concentration of 7.5×10¹⁰ pu/ml, the virus retained infectivity in all three formulations, albeit with some minor loss of infectivity. The loss was significantly less in the sucrose+VES+rHSA than with the other two formulations. These results show that simian adenovirus retained infectivity when formulated in the above-described compositions.

### Experiment 2: 7 days at 30° C. or 30 days at 25° C.

### Stability

Adenovirus was formulated at a concentration of 7.5×10¹⁰ pu/ml in 10 mM Tris pH 8.5, 5 mM NaCl, 0.024% polysorbate 80 (w/v), 10 mM histidine, 1 mM MgCl₂, with the addition of either: (1) 16% sucrose+0.05 mM VES; (2) 16% sucrose+0.05 mM VES+0.1% rHSA; or (3) 15% trehalose+0.05 mM VES+0.1% rHSA, as shown in the table below. The virus was either (1) stored at 4° C.; kept at a temperature of 30° C. for seven days; or kept at a temperature of 25° C. for 30 days. Stability was evaluated by analytical HPLC, PICOGREEN, and quantitative PCR in the presence and absence of benzonase under both accelerated stability conditions.

| | | | | | | | | qPCR¶ | | |
|---|---|---|---|---|---|---|---|---|---|---|
| ¶ Adeno- | | HPLC¶ | | | PICOGREEN¶ | | | (gE/ml)¶ | | |
| virus¶ | ¶ | (pu/ml)¶ | | | (%)¤ | | ¤ | (+) or (−)•benzonase | | |
| (pu/ml)¶ | Formu- | | 7 d¶ | 30 d¶ | | 7 d¶ | 30 d¶ | | 7 d¶ | 30 d¶ |
| ¤ | lation¤ | 4° C.¤ | 30° C.¤ | 25° C.¤ | 4° C.¤ | 30° C.¤ | 25° C.¤ | 4° C.¤ | 30° C.¤ | 25° C.¤ |
| ¶ | 16% sucrose¶ | ¶ | ¶ | ¶ | ¶ | ¶ | ¶ | ¶ | ¶ | (+) 5.28 × |
| 6 × 10¹⁰ | 0.05 mM | 6.64 × | 6.00 × | 5.16 × | 1.8%¤ | 2.5%¤ | 2.7%¤ | (+) 6.54 × | (+) 6.00 × | 10¹⁰¤ |
| pu/ml¤ | VES¶ | 10¹⁰¤ | 10¹⁰¤ | 10¹⁰¶ | | | | 10¹⁰¤ | 10¹⁰¤ | (−) 5.45 × |
| | ¤ | | | ¤ | | | | (−) 6.62 × | (−) 6.08 × | 10¹⁰¤ |
| | | | | | | | | 10¹⁰¤ | 10¹⁰¤ | |
| | 16% sucrose¶ | ¶ | ¶ | ¶ | ¶ | ¶ | ¶ | ¶ | ¶ | (+) 6.57 × |
| | 0.05 mM | 6.83 × | 6.54 × | 6.23 × | 1.8%¤ | 2.8%¤ | 3.8%¤ | (+) 7.06 × | (+) 6.91 × | 10¹⁰¤ |
| | VES¶ | 10¹⁰¤ | 10¹⁰¤ | 10¹⁰¤ | | | | 10¹⁰¤ | 10¹⁰¤ | (−) 6.87 × |
| | 0.1% rHSA¶ | | | | | | | (−) 6.75 × | (−) 6.51 × | 10¹⁰¤ |
| | ¤ | | | | | | | 10¹⁰¤ | 10¹⁰¤ | |
| | 15% trehalose¶ | ¶ | ¶ | ¶ | ¶ | ¶ | ¶ | ¶ | ¶ | (+) 5.15 × |
| | 0.05 mM | 6.79 × | 6.21 × | 5.24 × | 2.6%¤ | 4.1%¤ | 6.4%¤ | (+) 6.66 × | (+) 6.15 × | 10¹⁰¤ |
| | VES¶ | 10¹⁰¤ | 10¹⁰¤ | 10¹⁰¤ | | | | 10¹⁰¤ | 10¹⁰¤ | (−) 6.83 × |
| | 0.1% rHSA¤ | | | | | | | (−) 6.67 × | (−) 6.44 × | 10¹⁰¤ |
| | | | | | | | | 10¹⁰¤ | 10¹⁰¤ | |

The simian adenoviral vectors remained stable at 30° C. for seven days in all three formulations, albeit with a viral particle loss of 4-10%, as measured by analytical HPLC. As expected, the viral particle loss was more pronounced after 30 days at 25° C. Viral particle loss in sucrose+VES+rHSA was less than with the other two formulations.

As measured by PICOGREEN, the normalized average of DNA released from the viral capsid was somewhat higher after seven days at 30° C. and significantly higher after 30 days at 25° C. than at 4° C. in all three formulations, as expected. The percentage of released DNA was based on a regression between a freshly thawed control group and a control exposed to 60° C. for 30 min., which completely degraded the viral capsid.

As measured by qPCR, all three formulations also supported an acceptable thermostability profile. As expected, the number of genome equivalents per ml decreased somewhat after 30 days at 25° C. (benzo (+)) in all three formulations and after seven days at 30° C. in sucrose+VES and trehalose+VES+rHSA. Again, the best results were obtained with sucrose+VES+rHSA with only a 2% loss after seven days at 30° C. and only a 7% loss after 30 days at 25° C.

### Infectivity

Adenovirus was formulated at a concentration of 7.5×10¹⁰ pu/ml in 10 mM Tris pH 8.5, 5 mM NaCl, 0.02′% polysorbate 80 (w/v), histidine 10 mM, 1 mM MgCl₂, with the addition of either: (1) 16% sucrose (w/v)+0.05 mM VES; (2) 16% sucrose (w/v)+0.05 mM VES+0.1% rHSA (w/v); or (3) 15% trehalose (w/v)+0.05 mM VES+0.1% rHSA (w/v), as shown in the table below. The virus was either stored at 4° C. or kept 30° C. for seven days or 25° C. for 30 days. Infectivity was determined by measuring infectious units by FACS analysis.

| | | Infectivity | | | | | |
|---|---|---|---|---|---|---|---|
| | | Measured by IU | | | Infectivity | | |
| | | [log(IU/mL)] | | | Ratio | | |
| Adenovirus | | | 7 d | 30 d | | 7 d | 30 d |
| Concentration (pu/ml) | Formulation | 4° C. | 30° C. | 25° C. | 4° C. | 30° C. | 25° C. |
| 6 × 10¹⁰ pu/ml | 16% sucrose | 9.03 | 8.72 | 8.21 | 47 | 114 | 319 |
| | 0.05 mM VES | | | | | | |
| | 16% sucrose | 9.07 | 8.76 | 8.44 | 58 | 114 | 224 |
| | 0.05 mM VES | | | | | | |
| | 0.1% rHSA | | | | | | |
| | 15% trehalose | 9.07 | 8.76 | 8.25 | 58 | 107 | 293 |
| | 0.05 mM VES | | | | | | |
| | 0.1% rHSA | | | | | | |

As expected, some loss of viral infectivity was observed in all three formulations after seven days at 30° C. and after 30 days at 25° C. This loss was modest, about 3% loss of IU in all three formulations after seven days at 30° C. and about 7-9% after 30 days at 25° C. The infectivity ratio, i.e., the ratio of the number of infectious viral particles to the number of total viral particles, was determined by calculating the ratio of analytical HPLC to IU results.

### Example 6: Extrapolation of Stability Data

Data from an extrapolation of stability study were analyzed to estimate a loss of viral particle stability over time with statistical models that extrapolate viral particle loss over time. Both linear and first-order decay models were used and the results shown in the table below.

| | Formulation | | | | |
|---|---|---|---|---|---|
| | 10 mM Tris | | | | |
| | pH8.5, | | | | |
| | 5 mM | | | | |
| | NaCl, 0.02% | | | | |
| | polysorbate | | | Viral Particle Loss | |
| | 80 (w/v) | | Observed | Extrapolated to Two Years | |
| Viral Particle | 1 mM MgCl₂, | Adenoviral | Viral | Linear | First-order |
| Stability | with the | Concentration | Particle | model | decay model |
| (HPLC) | addition of: | (pu/ml) | Loss | (95% Cl) | (95% Cl) |
| | 15% Trehalose | 6 × 10¹⁰ | −4% | −15% | −7% |
| | 0.05 mM VES | | (six months) | ( −24%; −5%) | (−22%; −5%) |
| | 0.1% rHSA | 6 × 10⁹ | −1% | −2% | −2% |
| | | | (six months) | (−6%; +2%) | (−7%; +3%) |
| | 16% Sucrose | 6 × 10¹⁰ | −1% | −2% | −2% |
| | 0.05 mM VES | | (10 months) | (−4%; 0%) | (−3%; 0%) |
| | 0.1% rHSA | 6 × 10⁹ | No loss | 3% | 3% |
| | | | observed | (−4%; +11%) | (−5%; +12%) |
| | | | | Infectivity Decline | |
| | | | | extrapolated to two years | |
| Infectivity | | Adenoviral | Observed | Linear | First-order |
| of | | Concentration | Infectivity | Model | decay model |
| Transgene | Formulation | (pu/ml) | Decline | (95% Cl) | (95% Cl) |
| | 15% Trehalose | 6 × 10¹⁰ | −15% | −62% | −49% |
| | 0.05 mM VES | | (six months) | (−85%; −38%) | (−60%; −33%) |
| | 0.1% rHSA | 6 × 10⁹ | −15% | −58% | −47% |
| | | | (six months) | (−77%; −40%) | (−57%; −34%) |
| | 16% Sucrose | 6 × 10¹⁰ | −35% | −52% | −45% |
| | 0.05 mM VES | | (10 months) | (−65%; −39%) | (−53%; −36%) |
| | 0.1% rHSA | 6 × 10⁹ | −10% | −31% | −27% |
| | | | (six months) | (−49%; −13%) | (−40%; −13%) |

In the linear model, also known as zero order decay, the product characteristic, i.e., stability, decreases linearly with time and is calculated as Y=α₀+α₁×time. In the first order decay model, which is a linear model on log-transformed data, the product characteristic decay, i.e., the decay of stability, decreases over time and is calculated as Log(Y)=α₀+α₁×time. “Y” is a quality attribute, e.g., total particles or infectious particles. “α₀” is the intercept, i.e., the value of Y at initial time zero. “α₁” is the slope over time, i.e., the rate of degradation.

The following excipients were screened at 37° C. at a range of concentrations for their effect on infectivity, as shown in the table below.

| | Minimum | Maximum |
|---|---|---|
| Excipient | Conc. | Conc. |
| Histidine (mM) | 0 | 20 |
| Polysorbate 80 (%) | 0.025 | 0.125 |
| Sucrose (%) | 9 | 21 |
| MgCl₂ (mM) | 0 | 6 |
| VES (mM) | 0 | 0.25 |
| rHSA (%) | 0.02 | 0.3 |
| Methionine (mM) | 0 | 5 |

The main variables influencing infectivity were VES and MgCl₂. The greatest losses of infectivity were observed at high concentrations of MgCl₂ and low concentrations of VES. Metalloproteases present in the formulations could account for the detrimental effect of MgCl₂ on infectivity and can be investigated, e.g., by determining the effects of chelating agents, protease inhibitors, zymogram and optimizing the dose ranges of benzonase and rHSA, in the event either of these excipients is introducing a protease into the formulation. The detrimental effect of MgCl₂ on infectivity can potentially be overcome by increasing the concentration of MgCl₂, overloading the protease binding sites or, alternatively, removing MgCl₂ from the formulation.

### Example 7: Immunogenicity

Balb/c mice were immunized with either 3×10⁷ or 3×10⁶ viral particles of ChAd155-RSV formulated in (a) 10 mM Tris pH 8.5, 10 mM histidine, 5 mM NaCl, 16% sucrose (w/v), 0.024% polysorbate 80 (w/v), 1 mM MgCl₂, 0.05 mM VES and 0.1% rHSA or (b) a lyophilized formulation comprising 10 mM Tris pH 7.4, 10 mM histidine, 1 mM MgCl₂, 0.02% polysorbate 80, 25 mM NaCl and 8% sucrose.

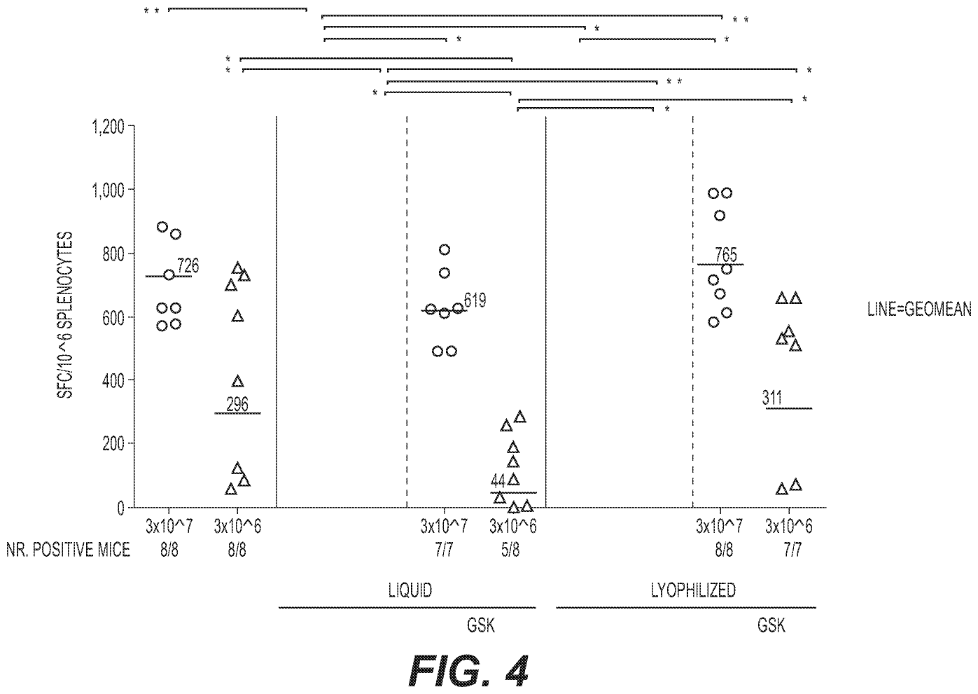

T-cell response was measured three weeks after the immunization by ex vivo IFN-gamma enzyme-linked immunospot (ELISpot) using the RSV-M2 immunodominant CD8 epitope and the results shown in FIG. 4. Each dot represents the response in a single mouse. Results are expressed as IFN gamma spot forming cells per million splenocytes. Horizontal lines represent the mean number of IFN-gamma SFC/million splenocytes for each dose group “geomean.”

FIG. 4 demonstrates that RSV-specific T cells were detected in the spleens of the mice immunized with either 3×10⁷ or 3×10⁶ viral particles. The liquid composition of the invention compared favorably with the lyophilized formulation. Therefore, compositions of the invention can be used as immunogenic simian adenoviral formulations.

### Example 8: Forced Degradation by Exposure to Metal and Light Oxidation

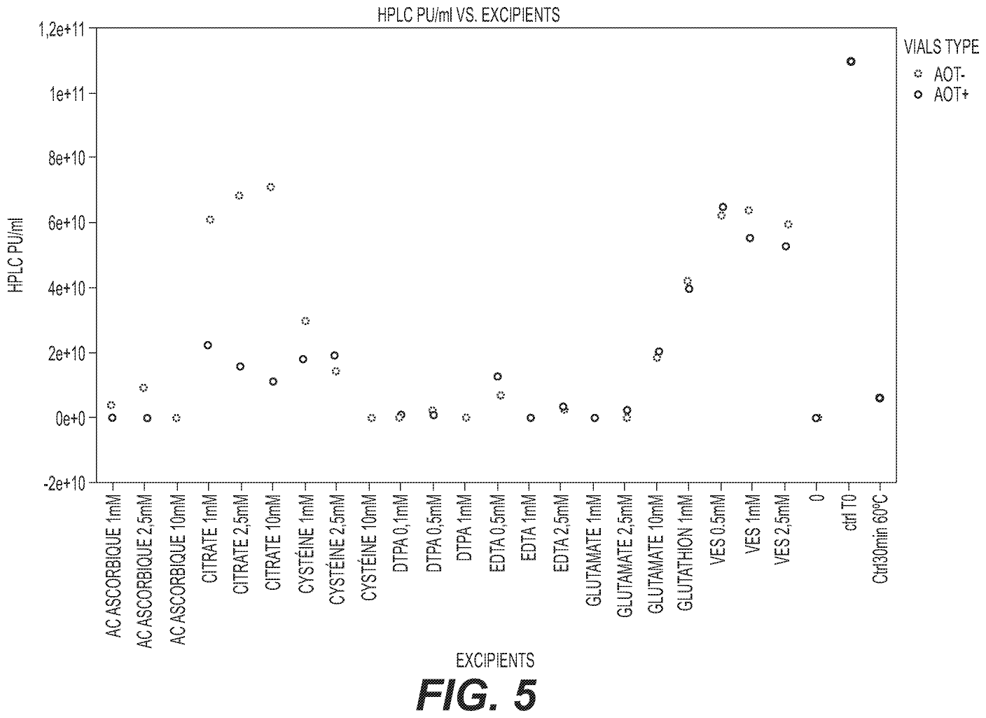

ChAd155-RSV was formulated at a concentration of 3×10¹⁰ or 2.8×10¹¹ viral particles per milliliter in 10 mM Tris pH 8.5, 25 mM NaCl, 8% sucrose (w/v), 0.02% polysorbate 80 (w/v) and 1 mM MgCl₂, then exposed to metal and light oxidation under Accelerated Oxidation Test (AOT) conditions for three, eight or ten days at 30° C., 37° C. or 42° C. Light irradiation conditions were 765 W/m² at 320-380 nm. The oxidation conditions exposure adenovirus formulations to a metal cocktail comprising 1.5 ppm each of Cu⁺², Ni⁺², Fe⁺³ and Cr⁺³ under the above light irradiation conditions. Excipients tested included ascorbic acid, citrate, cysteine, diethylenetriaminepentaacetic acid (DTPA), ethylenediaminetetraacetic acid (EDTA), glutamate and vitamin E succinate (VES). Stability was measured by analytical high performance liquid chromatography (HPLC) and the results were expressed as HPLC virus particle units per milliliter (pu/ml). Dotted-outlined dots indicate stability in the absence of the AOT conditions (AOT (−) vials) and solid-outlined dots indicate stability in the presence of the AOT conditions (AOT (+) vials). As shown in FIG. 5, the best results were obtained when VES was present; stability remained high in both the presence and absence of metal and light-induced oxidation.

1.-48. (canceled)

49. An aqueous composition comprising a simian adenoviral vector, a Tris buffer, an amorphous sugar, histidine, NaCl, a surfactant, Vitamin E succinate and recombinant human serum albumin.

50. The composition of claim 49 wherein the amorphous sugar is sucrose.

51. The composition of claim 50 wherein the concentration of sucrose is less than about 30% (w/v), for example, less than about 25% (w/v), about 10% to about 20% (w/v) or about 16% (w/v).

52. The composition of claim 49 wherein the amorphous sugar is trehalose.

53. The composition of claim 52 wherein the concentration of trehalose is about 1% to about 30% (w/v), for example, about 10% to about 20% (w/v), about 15% to about 16% (w/v).

54. The composition of claim 49 wherein the concentration of Vitamin E succinate is about 0.001 mM to about 0.5 mM.

55. The composition of claim 49 wherein the concentration of recombinant human serum albumin is about 0.01% to about 1.0%.

56. The composition of claim 49 wherein the Tris buffer is pH 6-10 at a concentration of about 1.0 mM to about 25 mM.

57. The composition of claim 49 wherein the concentration of histidine is less than about 15 mM.

58. The composition of claim 49 wherein the concentration of sodium chloride is less than about 30 mM.

59. The composition of claim 49 wherein the surfactant is selected from a poloxamer surfactant and a polysorbate surfactant.

60. The composition of claim 59 wherein the surfactant is polysorbate 80 at a concentration of about 0.01% (w/v) to about 0.05% (w/v).

61. The composition of claim 49 further comprising a bivalent metal salt.

62. The composition of claim 61 wherein the bivalent metal salt is selected from MgCl₂, CaCl₂ and MgSO₄.

63. The composition of claim 62 wherein the bivalent metal salt is MgCl₂ at a concentration of about 0.1 mM to about 10.0 mM.

64. The composition of claim 49 wherein the concentration of the simian adenoviral vector is between about 1×10⁶ and about 1×10¹² viral particles per milliliter.

65. The composition of claim 49 wherein the simian adenoviral vector comprises a transgene.

66. The composition of claim 65 wherein the transgene is an immunogenic transgene.

67. The composition of claim 49 wherein the simian adenoviral vector is replication defective.

68. The composition of claim 49 wherein the simian adenoviral vector is replication competent.

69. An aqueous composition comprising a simian adenoviral vector, a Tris buffer, an amorphous sugar, histidine, NaCl, a surfactant, Vitamin E succinate and recombinant human serum albumin wherein the simian adenoviral vector is stable for at least six months at 4° C.

70. An aqueous composition comprising a simian adenoviral vector, a Tris buffer, an amorphous sugar, histidine, NaCl, a surfactant, Vitamin E succinate and recombinant human serum albumin wherein the simian adenoviral vector is stable for at least fifteen days at 25° C.

71. An aqueous composition a simian adenoviral vector, a Tris buffer, an amorphous sugar, histidine, NaCl, a surfactant, Vitamin E succinate and recombinant human serum albumin wherein the simian adenoviral vector is stable for at least four days at 30° C.

72. A method of making an aqueous composition comprising a simian adenoviral vector, a Tris buffer, an amorphous sugar, histidine, NaCl, a surfactant, Vitamin E succinate and recombinant human serum albumin comprising
(a) producing a simian adenovirus in a host cell; and
(b) combining the adenovirus with one or more excipients;
wherein the adenovirus is stable (i) at +2−+8° C. for at least six months; or (ii) at 25° C. for at least fifteen days; or (iii) at 30° C. for at least four days.

73. A method of eliciting an immune response in a mammalian subject comprising administering to the subject the composition of claim 66.
